(19) United States
(12) Patent Application Publication
(10) Pub. No.: US 2022/0409420 A1
(43) Pub. Date: Dec. 29, 2022

(54) EASY EMPTY URINE COLLECTION BAGS

(71) Applicant: Hollister Incorporated, Libertyville, IL (US)

(72) Inventors: Jerome A. Henry, Castlebar (IE); Daniel E. O'Brien, Calry (IE); Martin McMenamin, Lifford (IE); Gerard P. Grimes, Ballinrobe (IE); Vincent Naughton, Sligo (IE)

(21) Appl. No.: 17/778,976

(22) PCT Filed: Nov. 9, 2020
(86) PCT No.: PCT/US2020/059727

§ 371 Date: May 23, 2022

Related U.S. Application Data
(60) Provisional application No. 62/939,848, filed on Nov. 25, 2019.

(51) Int. Cl. A61F 5/44; A61M 1/00

(52) U.S. Cl. CPC A61F 5/4407; A61M 1/69; A61M 1/70

(57) ABSTRACT

Urinary catheter collection bags and methods for using the same are provided. The collection bags allow users to more easily, cleanly and comfortably open and empty the bags.

The present application claims the benefit of and priority to U.S. Provisional Application No. 62/939,848, filed Nov. 25, 2019, which is hereby incorporated herein by reference.

### DESCRIPTION

### Technical Field

The present disclosure generally relates to urinary catheters. More particularly, the present disclosure relates to urinary catheter collection bags.

### Background

Catheters are used to treat many different types of medical conditions and typically include an elongated tube that is inserted into and through a passageway or lumen of the body. Catheters, and in particular intermittent catheters, are commonly used by those who suffer from various abnormalities of the urinary system, such as urinary incontinence. Urinary catheters generally comprise a tube with two ends. A first end has a catheter tip that may be inserted into a user's urethra. A second end generally has a drainage member that is used to help facilitate drainage of urine from the tube. Some urinary catheters include a urine collection bag connected to the drainage member of the catheter. When the bag is connected to the catheter, the catheter directs urine to flow into the attached collection bag.

After catheterization, the user typically empties the urine out of the bag into a toilet. The user then disposes the bag in a trash receptacle. Many catheter users have neurological conditions like stroke, Multiple Sclerosis (MS), or acute spinal cord injuries (SCI). Consequently, some catheter users may have limited dexterity, which in some instances can make it challenging for such users to open a urine collection bag. Accordingly, there is a need for urinary catheter collection bags that allow users to easily, cleanly and comfortably empty the bag themselves.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a urinary catheter collection bag comprises a first flexible wall having a perimeter, and a second flexible wall having a perimeter. A perimeter seal joins at least a portion of the perimeter of the first flexible wall to at least a portion of the perimeter of the second flexible wall, to define a cavity between the first and second flexible walls. The seal defines a boundary of the collection bag. A tear starting member extends beyond the boundary of the bag.

In another aspect, a surface of a urinary catheter collection bag includes a drain hole. The drain hole is covered by a water soluble cover member. The water soluble cover member is configured to dissolve after being submerged in a solution. After the water soluble cover member is dissolved, the drain hole is uncovered.

### BRIEF DESCRIPTION OF THE DRAWINGS

FIG. 1 is a perspective view of an embodiment of a urinary catheter collection bag in accordance with the present disclosure;

FIG. 1A is a rear perspective view of the urinary catheter collection bag of FIG. 1;

FIG. 2 is a perspective view of the bag of FIG. 1 torn open and being emptied;

FIG. 3 is a perspective view of an embodiment of a urinary catheter collection bag in accordance with the present disclosure;

FIG. 4 is a perspective view of the bag of FIG. 3 with an exposed drain hole.

### DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter as defined in the accompanying claims.

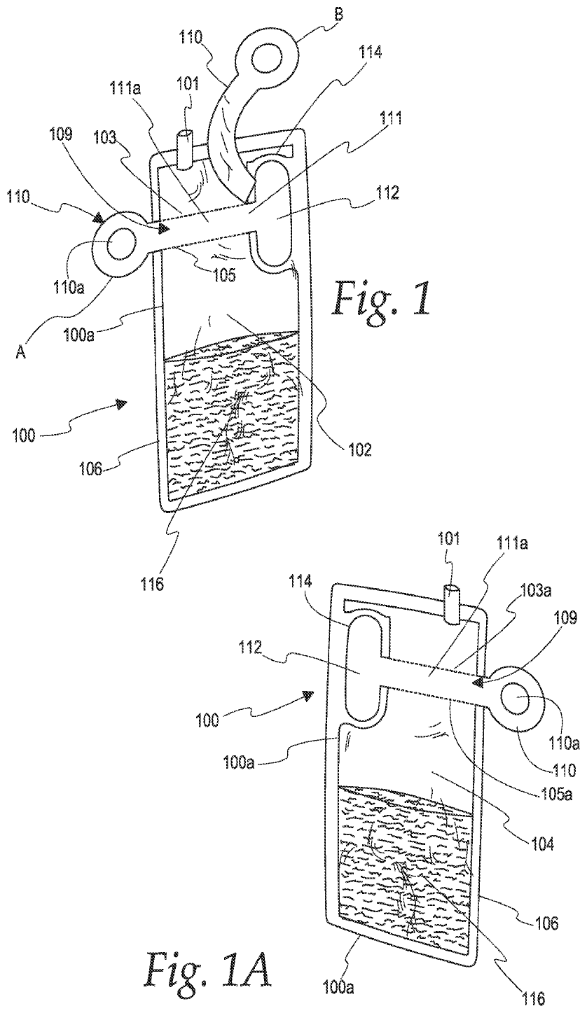

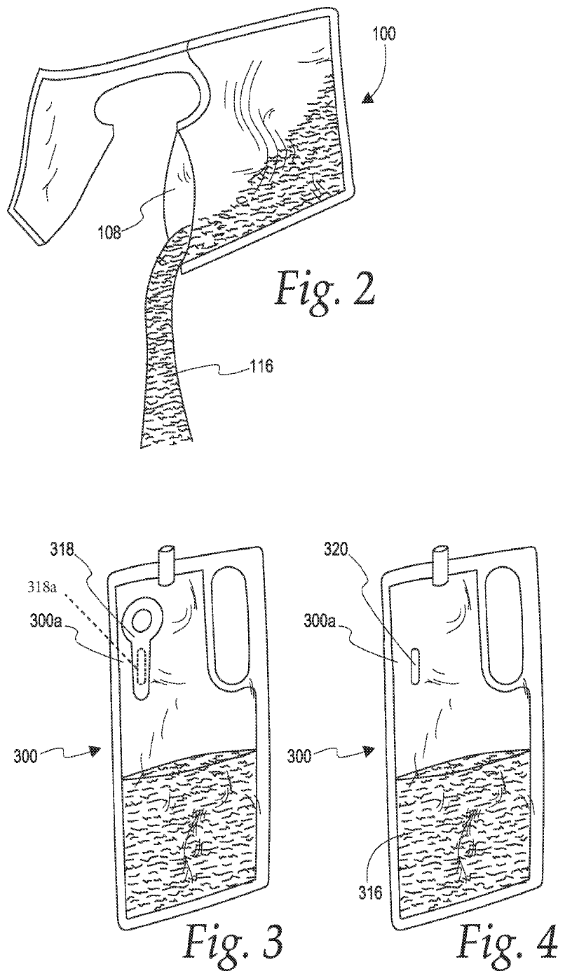

Urinary catheter collection bags according to the present disclosure and their individual components may be variously configured without departing from the scope of the present disclosure, but in one exemplary embodiment, a urinary catheter collection bag 100 is configured as shown in FIG. 1. The collection bag 100 comprises a first flexible wall 102 having a perimeter, and a second flexible wall 104 (FIG. 1A) having a perimeter. A perimeter seal 106 joins at least a portion of the perimeter of the first flexible wall 102 to at least a portion of the perimeter of the second flexible wall 104 to define a cavity 108 (FIG. 2) between the first flexible wall 102 and second flexible wall 104. The seal 106 defines a boundary 100a of the collection bag 100. In the illustrated embodiment, the first flexible wall 102 and the second flexible wall 104 are defined by separate sheets of material that are sealed to one another. In another embodiment, the first and second walls 102 and 104 may be formed from a single sheet of material that is folded over to define the first and second walls. The walls 102 and 104 are connected at the fold located at bottom of the walls and are sealed together along the sides and tops of walls to form the collection bag 100. Additionally, the bag 100 includes a port or opening 101 for connection to a catheter. For example, the port 101 may be connected to a drainage member of a catheter.

The bag 100 also includes a tear starting member 110 that extends beyond the boundary 100a of the bag 100. The tear starting member 110 may be ring shaped and/or may include a hole 110a on an end that extends beyond the boundary 100a of the bag 100. In alternative embodiments, the tear starting member 110 may have any suitable shape and size.

As shown in FIGS. 1 and 1A, in one embodiment the tear starting member 110 may extend from a tearable portion 109 located in the first and/or second walls 102 and 104 of the urine collection bag 100. In the illustrated embodiment, the tearable portion 109 may include tear strips 111 and 111a located in the first wall 102 and second wall 104, respectively. As shown in FIG. 1, the tear strip 111 may be defined by a first top tear line 103 and a second bottom tear line 105. Tear lines 103 and 105 extend across the first wall from the tear starting member 110 toward the opposite edge of the bag 100. Similarly and as shown in FIG. 1A, the tear strip 111a may be defined by a first top tear line 103a and a second bottom tear line 105a. Tear lines 103a and 105a extend across the second wall 104 from the tear starting member 110 toward the opposite edge of the bag 100. When the tearable portion 109 includes tear strips 111 and 111a, the tear strips may be opposed to one another. Furthermore, the tear starting member 110 may be integral or of unitary construction with the tearable portion 109. Alternatively, the tear starting member 110 may be attached to the tearable portion 109 by, for example, adhesive or welding.

Turning back to FIG. 1, this figure shows the tear starting member 110 and the tear portion 109 being moved from a first position A to a second position B to open the urine collection bag 100 for the drainage of urine therefrom. In use, the user grasps the tear starting member 110 and pulls it to tear the tear portion 109 of the collection bag. Tear lines 103 and 105 selectively direct tearing of the first flexible wall 102, and tear lines 103a and 105a selectively direct tearing of the second flexible wall 104. When the tear starting member 110 is in the second position B, it has traveled the full length of the first and second tear lines (103, 103a, 105, 105a) of each of the walls and is removable from the bag 100. When the starting member 110 is removed from the bag 100 the cavity 108 (shown in FIG. 2) is exposed and the bag's contents (the fluid within the bag) 116 may be emptied by the user. The tear lines 103, 103a, 105, and 105a may be score lines. In another embodiment, the tearable portion may be made from directional tear polymers, which direct tearing in a desired direction. For example, the directional tear polymers may direct tearing across the wall of the collection bag. In one embodiment, the tear lines of each wall are defined by directional tear polymers. In another embodiment tear lines 103, 103a, 105 and 105a may be defined by directional tear tape.

Additionally, the tear lines 103, 103a, 105, 105a may extend to a cutout 112 in the bag 100. The cutout 112 may comprise a terminus for the tear lines 103, 103a, 105, 105a. Additionally, the cutout 112 may form a handle 114 for gripping the bag 100.

The bag 100 may have a generally rectangular shape having a first side boundary and a second side boundary and the tear starting member 110 may extend from one of the side boundaries. Additionally, the first tear lines 103, 103a and the second tear lines 105, 105a, of the respective walls, may extend from one of the side boundaries toward the other of the side boundaries. For example, in an embodiment the first tear lines 103, 103a and second tear lines 105, 105a may both extend from the first side boundary to the second side boundary.

FIG. 2 shows the urinary catheter bag 100, after the tear starting member has been removed. As shown in FIG. 2, the bag 100 is open and the cavity 108 is exposed. A user may empty the bag 100 by draining the contents 116 through the cavity 108. The contents 116 may be drained into a toilet or any other appropriate receptacle.

FIG. 3 illustrates an embodiment of a urinary collection bag 300, which has similar structures to those described in FIGS. 1 and 2. However, the urine collection bag 300 may be any other suitable bag for collecting urine. The bag 300 includes a surface 300a, where the surface of the bag 300a includes a drain hole 320 (visible in FIG. 4). As shown in FIG. 3, the drain hole is covered by a cover member 318. In one embodiment, the cover member 318 is water soluble and may be attached to the bag 300 by an adhesive or may be integral to the bag 300. When made of a water soluble material, the cover member 318 is configured to dissolve after coming into contact with water or a solution containing water such that the drain hole is uncovered after the cover member is dissolved. Thus, when water touches the water soluble cover member the drain hole is exposed. FIG. 4 shows the bag 300 after the water soluble cover member 318 has dissolved and the drain hole 320 is exposed. When the drain hole 320 is exposed, the contents 316 of the bag 300 may exit through the drain hole 320 and into the toilet, or another appropriate receptacle. Thus a user may empty the bag 300, such that fluid within the bag (the contents) 316 may drain through the drain hole 320.

FIG. 3 also shows a water soluble member 318a situated behind the cover member 318. The water soluble member 318a dissolves, creating a drain hole. The covering member 318 may be made of materials including polyethylene (PE), or ethylene vinyl acetate (EVA). The covering member 318 sits in front of the water soluble cover member 318a. Once the water soluble cover member 318a is dissolved, the covering member 318 is removed to uncover the drain hole to allow the user to drain the bag in a toilet or appropriate receptacle. The water soluble member 318a may be made out of a water soluble material including, but not limited to, paper, polyvinyl alcohol (PVOH), polyvinyl acetate (PVA), or ethylene vinyl acetate (EVA).

In another embodiment, there is no water soluble cover member. Instead, the bag is pierced forming a drain hole. A self-adhesive cover member is placed above the drain hole in order to obstruct/stop the flow of urine out of the bag. The self-adhesive cover member is removed, uncovering the drain hole when the bag is ready to be drained in a toilet or other appropriate receptacle.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

What is claimed is:

1. A urinary catheter collection bag, comprising
a first flexible wall having a perimeter;
a second flexible wall having a perimeter;
a perimeter seal joining at least a portion of the perimeter of the first flexible wall to at least a portion of the perimeter of the second flexible wall to define a cavity between the first and second flexible walls, the seal defining a boundary of the collection bag; and
a tear starting member extending beyond the boundary of the bag.

2. The urinary collection bag of claim 1, wherein the tear starting member is integral with the first and second flexible walls.

3. The urinary collection bag of claim 1, wherein the tear starting member extends from a tearable portion of the urine collection bag.

4. The urinary collection bag of claim 1, wherein the first flexible wall includes a first top tear line and a second bottom tear line extending from the tear starting member and the second flexible wall includes a top tear line and a bottom tear line extending from the starting member.

5. The urinary collection bag of claim 4, wherein the tear lines selectively direct tearing of the first and the second flexible walls.

6. The urinary collection bag of claim 4, wherein the tear lines are score lines.

7. The urinary collection bag of claim 4, wherein the tear lines are defined by a directional tear polymer.

8. The urinary collection bag of claim 4, wherein the first top and second bottom tear lines are defined by a tear tape.

9. The urinary collection bag of claim 4, wherein tear lines extend to a cutout in the bag.

10. The urinary collection bag of claim 9, wherein the cutout comprises a terminus for the tear lines.

11. The urinary collection bag of claim 9, wherein the cutout forms a handle for gripping the bag.

12. The urinary collection bag of claim 4, wherein the bag has a generally rectangular shape having a first side boundary and second side boundary and the tear starting member extends from one of the side boundaries.

13. The urinary collection bag of claim 12, wherein the first and second tear lines extend from the one of the side boundaries toward the other of the side boundaries.

14. A urinary catheter collection bag, wherein a surface of the bag includes a drain hole wherein the drain hole is covered by a water soluble cover member, the water soluble cover member being configured to dissolve after being submerged in a solution such that the drain hole is uncovered after the cover member is dissolved.

15. A method for emptying a urinary catheter collection bag, comprising the steps of:
actuating a starting member extending beyond a first boundary of the bag;
moving the starting member along a score line toward a cutout, such that the bag tears along the score line;
stopping the tear at the cutout, such that the bag does not tear past a second boundary and the tear forms an opening along a portion of the bag;
emptying the bag, such that fluid within the bag may drain through the opening.
